(19) United States
(12) Patent Application Publication
(10) Pub. No.: US 2011/0313319 A1
(43) Pub. Date: Dec. 22, 2011

(54) MEASURING METHOD FOR THE CONCENTRATION OF INTRAGASTRIC CONTENTS

(75) Inventor: Yuan-Soon Ho, Hu-Ko (TW)

(73) Assignee: SAND COUNTY BIOTECHNOLOGY, Inc., Hu-Ko (TW)

(21) Appl. No.: 13/220,153

(22) Filed: Aug. 29, 2011

Related U.S. Application Data
(63) Continuation-in-part of application No. 12/347,215, filed on Dec. 31, 2008.

(30) Foreign Application Priority Data
Nov. 7, 2008 (TW) 097142987

(51) Int. Cl. A61B 5/00

(52) U.S. Cl. 600/582

(57) ABSTRACT

A measuring method for the concentration of intragastric objects wherein a first concentration of a feeding liquor is measured. The intragastric objects and concentration thereof of the testee are measured to obtain the second concentration value. The feeding liquor of predefined volume is filled into the stomach of the testee. The intragastric contents are mixed and diluted to form an intragastric mixture. The stomach is then given time to digest the feeding liquor. The intragastric mixture is extracted and measured to obtain the third concentration value. The volume of intragastric objects is calculated as a relative value, so a relative volume could be measured by the volume of intragastric mixture and feeding liquor. According to the changes of said relative value with respect to the concentration and volume, the tester could judge the absorbing state of the testee's stomach as a basis of continuous feeding and feeding dose.

### CROSS-REFERENCE TO RELATED U.S. APPLICATIONS

The present application is a continuation-in-part of U.S. patent application Ser. No. 12/347,215, filed on Dec. 31, 2008, and entitled “Measuring Device and Method of Using the Device to Measure Concentration of Intragastric Contents”, presently pending.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to a measuring method for the concentration of intragastric contents, and more particularly to an innovative one which is used to measure the concentration of intragastric contents with an electro-chemical specimen and detector.

2. Description of Related Art Including Information Disclosed Under 37 CFR 1.97 and 37 CFR 1.98

Conventionally, the remaining volume of a patient's stomach was measured by adsorbing the stomach with a nasogastric tube. Before nasogastric tube feeding for the patients with higher remaining volume of stomach, the possible symptoms against the patients or potential hazards arising from inhalation pneumonia must be considered. Up to now, many physicians, nurses and nutritionists still take the volume of stomach remainders as the basis of deciding whether to stop intestinal feeding.

However, this cannot identify efficiently the extra stomach volume arising from emptying of external feeds or from endogenous excretion. Thus, the way of detecting the stomach's emptying conditions with the volume of stomach remainders is often restrained by the poor sensitivity and failure of fully adsorbing the intragastric contents. Moreover, the volume of adsorbed stomach remainders is not sensitive in the operation, and it is also impossible to distinguish the residual intestines composition from numerous endogenous secretions.

In view of the existing problems in monitoring and measuring the stomach emptying conditions as mentioned above, a refractometer has been developed to measure the refraction value of the intragastric food for examining the stomach emptying conditions. Said refractometer is used to check the concentration of fluids with the light refraction principle, of which the refraction value of foods in the gastric juice is proportional to the food concentration. When the food concentration prior to tube feeding is set as 100%, the refraction value is measured with the refractometer. After feeding for a period of time, 2-3 cc gastric juice is extracted to measure the refraction value, and it is converted into the final food concentration. Next, 30 cc physiological salt solution is filled into the stomach via a nasogastric tube, and fully mixed with the gastric juice, and then 2-3 cc is extracted to measure the concentration of diluted gastric juice. Finally, the volume of the gastric juice is calculated. However, it is found from actual experience that the typical measuring method for stomach emptying is suitable only provided that the remainders in the stomach are in liquid state. If solid substances are contained therein, the light refraction effect will be influenced, thus yielding possibly greater error of measurement results of the refractometer in disagreement with the standards.

It is observed that the saliva and intestinal juice from spontaneous secretion is not a clear solution, and some solid substances are contained in the gastrointestinal tract in the human body. So, the typical measurement method may still lead to greater error and poor accuracy, even if the stomach remainders are in a fully liquid state.

Thus, to overcome the aforementioned problems of the prior art, it would be an advancement if the art to provide an improved structure that can significantly improve the efficacy.

Therefore, the inventor has provided the present invention of practicability after deliberate experimentation and evaluation based on years of experience in the production, development and design of related products.

### BRIEF SUMMARY OF THE INVENTION

Based on the “measuring method for the concentration of intragastric objects” of the present invention wherein a measuring device is mainly used to measure the concentration of feeding liquor, intragastric contents and intragastric mixture before and after feeding, so the tester could judge the absorbing state of the testee's stomach according to the changes of relative concentration as a basis of continuous feeding and feeding dose. With this method, it is possible to measure more accurately and conveniently the concentration of intragastric contents and stomach emptying conditions with better applicability.

Based on the unique aspect of the present invention wherein “the measuring device for the concentration of intragastric objects” is mainly composed of an electro-chemical specimen and a detector, it is possible to measure directly the concentrations of saccharide, protein and amino acid in the intragastric objects, so the measurement is not affected by any solid substance, enabling the concentration of intragastric objects and stomach emptying conditions to be measured more accurately and conveniently.

Additionally, it is the first time that electro-chemical specimen technology has been applied to measure the concentration of intragastric objects, notwithstanding it is already used in other medical applications (e.g. measurement of blood sugar). It is also found that it can efficiently resolve the problem of greater interference error of solids arising from typical refractometer measurement, helping to improve substantially the measurement accuracy of stomach emptying, control the feeding dose of patients and guarantee the recovery process.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 shows a block diagram of the measurement steps in the measuring method of the present invention.

FIG. 2 shows an exploded perspective view of the preferred embodiment of measuring device of the present invention.

FIG. 3 shows an exploded perspective view of the electro-chemical specimen of measuring device of the present invention.

FIG. 4 shows a schematic view of the measurement steps of the measuring device.

FIG. 5 shows another schematic view of the measurement steps of the measuring device.

### DETAILED DESCRIPTION OF THE INVENTION

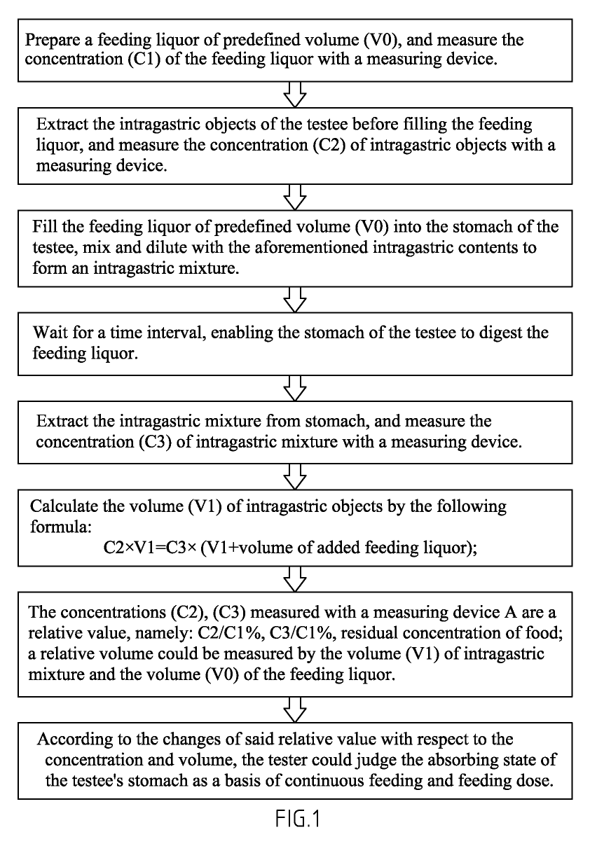

FIG. 1 depicts a preferred embodiment of measuring method of the present invention for the concentration of intragastric contents, which, however, are provided for only explanatory objective for patent claims; the measuring method for the concentration of intragastric contents may contain the following steps:

a) prepare a feeding liquor of predefined volume (V0), and measure the concentration (C1) of the feeding liquor with a measuring device A;

b) extract the intragastric objects of the testee before filling the feeding liquor, and measure the concentration (C2) of intragastric objects with a measuring device A;

c) fill the feeding liquor of predefined volume (V0) into the stomach of the testee, mix and dilute with the aforementioned intragastric contents to form an intragastric mixture;

d) wait for a time interval, enabling the stomach of the testee to digest the feeding liquor;

e) extract the intragastric mixture from stomach, and measure the concentration (C3) of intragastric mixture with a measuring device A;

f) calculate the volume (V1) of intragastric objects by the following formula:

C2×V1=C3×(V1+volume of added feeding liquor);

g) the concentrations (C2), (C3) are measured with a measuring device A are a relative value, namely: C2/C1%, C3/C1%, residual concentration of food; a relative volume could be measured by the volume (V1) of intragastric mixture and the volume (V0) of the feeding liquor;

h) according to the changes of said relative value with respect to the concentration and volume, the tester could judge the absorbing state of the testee's stomach as a basis of continuous feeding and feeding dose.

Of which, the concentration to be measured with a measuring device A refers to the concentration of either of saccharide, protein and amino acid.

Of which, the waiting time after filling said feeding liquor into the stomach of testee ranges between 0.1 h and 3 h.

When it is intended to fill the feeding liquor again, intragastric contents are firstly extracted from the stomach of testee, and the concentration (C4) of the intragastric contents is measured with a measuring device A, then a relative concentration is obtained for the tester by comparing the concentration (C4) with the concentration (C3) obtained in Step (E).

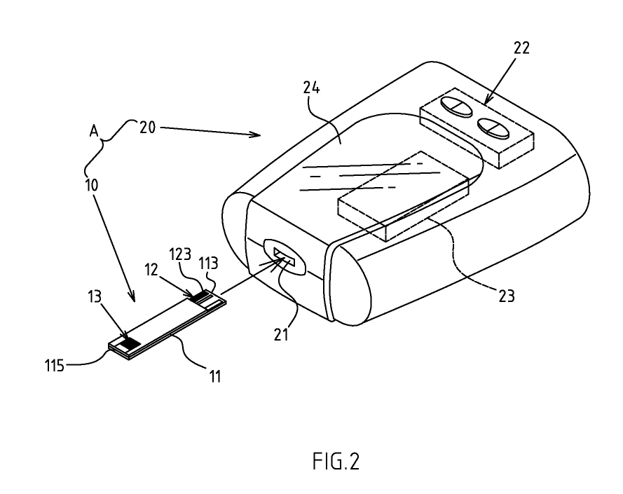

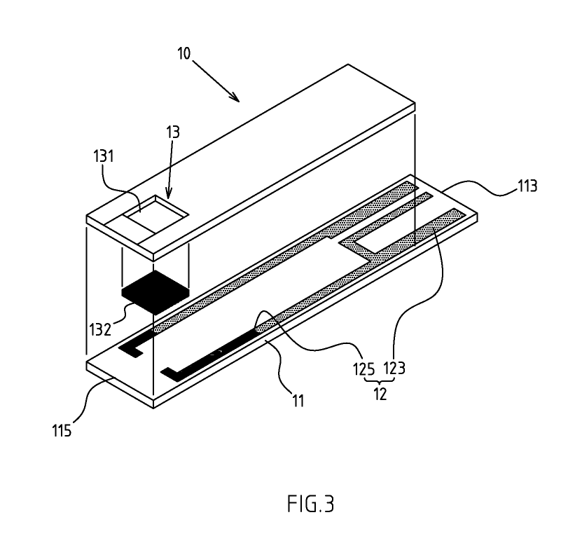

Referring also to FIGS. 2 and 3, the measuring device A for the concentration of intragastric contents comprising:

An electro-chemical specimen 10 containing: a board type insulating substrate 11, with an insertion end 113 and a sensing end 115; an electrode unit 12, containing a detection zone 123 and a reaction zone 125; the detection zone 123 corresponds to the insertion end 113 of the board type insulating substrate 11, whilst the reaction zone 125 corresponds to the sensing end 115 of the board type insulating substrate 11; a reaction portion 13, assembled onto the sensing end 115 of the board type insulating substrate 11, and aligned with the reaction zone 125 of the electrode unit 12; moreover, the reaction portion 13 is provided with an intragastric object inlet 131 and a chemical reaction zone 132.

A detector 20 containing: a measurement slot 21, used for mating the insertion end 113 of the electro-chemical specimen 10; a voltage generation unit 22, used to generate a preset voltage; a detection processing unit 23, used to detect the current signals generated by the electro-chemical specimen 10 containing intragastric objects within a time cycle, and interrelate the current signals with the concentration of the intragastric objects; a measurement display screen 24, used to display the measurement results (i.e. concentration) of the detector 20.

Of which, the measuring device A is used to measure the concentrations of saccharide, protein and amino acid in the intragastric objects.

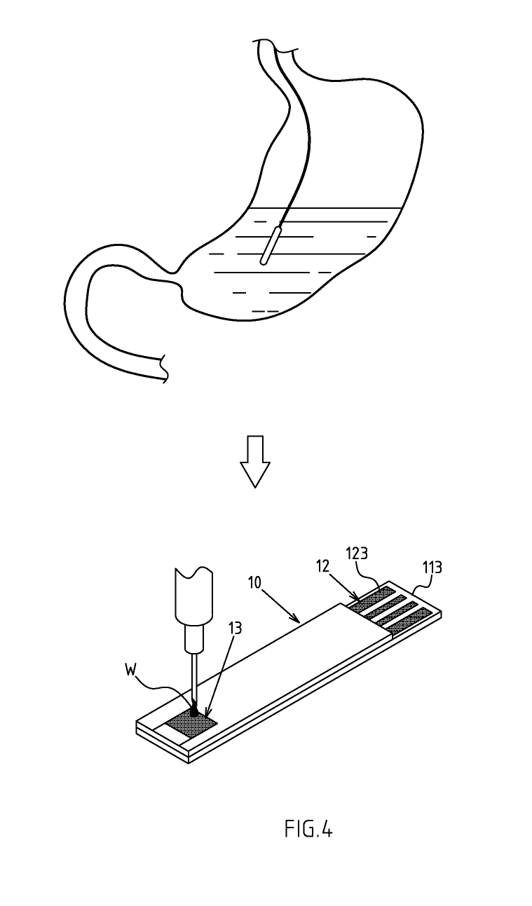

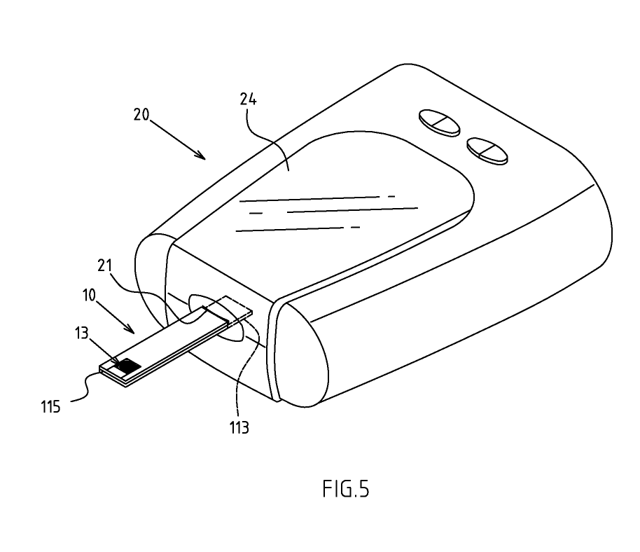

Based on the structural design of the aforementioned measuring device A, the intragastric objects are detected and measured as shown in FIG. 4, wherein the intragastric objects W are dripped into the reaction portion 13 from the intragastric object inlet 131 of the electro-chemical specimen 10 (or by syringe), and the insertion end 113 of the electro-chemical specimen 10 is inserted into the measurement slot 21 of the detector 20 (shown in FIG. 5). Then, the voltage generation unit 22 of the detector 20 generates a preset voltage to the electrode unit 12 of the electro-chemical specimen 10. In such a case, the saccharide, protein and amino acid, etc, in the intragastric objects W will yield chemical reaction with the chemical reaction zone 132 in the reaction portion 13 so as to generate electrons. The electrons will be accumulated in the reaction zone 125 of the electrode unit 12, so the detection processing unit 23 of the detector 20 could detect the amount of electrons in the reaction zone 125. The current value is computed by the built-in conversion formula of the detection processing unit 23, making it possible to calculate the concentration of the intragastric objects W and display it on the measurement display screen 24.

1. A measuring method for the concentration of intragastric objects comprising:
a) prepare a feeding liquor of predefined volume (V0), and measure the concentration (C1) of the feeding liquor with a measuring device;
b) extract the intragastric objects of the testee before filling the feeding liquor, and measure the concentration (C2) of intragastric objects with a measuring device;
c) fill the feeding liquor of predefined volume (V0) into the stomach of the testee, mix and dilute with the aforementioned intragastric contents to form an intragastric mixture;
d) wait for a time interval, enabling the stomach of the testee to digest the feeding liquor;
e) extract the intragastric mixture from stomach, and measure the concentration (C3) of intragastric mixture with a measuring device;
f) calculate the volume (V1) of intragastric objects by the following formula:
C2×V1=C3×(V1+volume of added feeding liquor);
g) the concentrations (C2), (C3) measured with a measuring device are a relative value, namely: C2/C1%, C3/C1%, residual concentration of food; a relative volume could be measured by the volume (V1) of intragastric mixture and the volume (V0) of the feeding liquor;
h) according to the changes of said relative value with respect to the concentration and volume, the tester could judge the absorbing state of the testee's stomach as a basis of continuous feeding and feeding dose.

2. The method defined in claim 1, wherein the concentration to be measured with said measuring device refers to the concentration of either of saccharide, protein and amino acid.

3. The method defined in claim 1, wherein the waiting time after filling said feeding liquor into the stomach of testee ranges between 0.1 h and 3 h.

4. The method defined in claim 1, wherein if it is intended to fill the feeding liquor again, intragastric contents are firstly extracted from the stomach of testee, and the concentration (C4) of the intragastric contents is measured with a measuring device, then a relative concentration is obtained for the tester by comparing the concentration (C4) with the concentration (C3) obtained in Step (E).

5. The method defined in claim, wherein the structure of said measuring device comprises:
an electro-chemical specimen, containing: a board type insulating substrate with an insertion end and a sensing end; an electrode unit, containing a detection zone and a reaction zone; the detection zone corresponds to the insertion end of the board type insulating substrate, whilst the reaction zone corresponds to the sensing end of the board type insulating substrate; a reaction portion, assembled onto the sensing end of the board type insulating substrate, and aligned with the reaction zone of the electrode unit; moreover, the reaction portion is provided with an intragastric object inlet and a chemical reaction zone;
a detector, containing: a measurement slot, used for mating the insertion end of the electro-chemical specimen; a voltage generation unit, used to generate a preset voltage; a detection processing unit, used to detect the current signals generated by the electro-chemical specimen containing intragastric objects within a time cycle, and interrelate the current signals with the concentration of the intragastric objects; a measurement display screen, used to display the measurement results of the detector.
